(19) United States
(12) Patent Application Publication
(10) Pub. No.: US 2017/0157112 A1
(43) Pub. Date: Jun. 8, 2017

(54) HIF INHIBITORS

(71) Applicant: UCL BUSINESS PLC, London (GB)

(72) Inventors: Margaret Ashcroft, Cambridge, Cambridgeshire (GB); Keith Jones, Sutton, Surrey (GB)

(73) Assignee: UCL Business PLC, London (GB)

(21) Appl. No.: 15/039,358

(22) PCT Filed: Nov. 25, 2014
(86) PCT No.: PCT/GB2014/053475

§ 371 Date: May 25, 2016

(30) Foreign Application Priority Data
Nov. 26, 2013 (GB) 1320835.0

(51) Int. Cl. A61K 31/4745

(52) U.S. Cl. CPC A61K 31/4745; A61K 2121/00

(57) ABSTRACT

The invention provides inhibitors of hypoxia-inducible factors (HIF), and their use in the prevention or inhibition of diseases characterised by abnormal HIF activity or levels, such as tumour progression, and the treatment of cancer. The invention encompasses pharmaceutical compositions with a mechanism of action for blocking elevated HIF activity in diseases, such as cancer.

The invention relates to hypoxia-inducible factors (HIF), and particularly, although not exclusively, to the inhibition of HIF activity. The invention extends to inhibitors of HIF activity, and their use in the prevention or inhibition of diseases characterised by abnormal HIF activity or levels, such as tumour progression, and the treatment of cancer. The invention encompasses pharmaceutical compositions and methods of treating diseases characterised by elevated HIF activity, such as cancer.

The hypoxia-inducible factor (HIF) transcriptional complex is involved in tumour progression by up-regulating key genes involved in metabolic adaptation, glycolysis (glucose transporters, GLUT1 and glycolytic enzymes), proliferation (insulin-like growth factors 1 and 2) and angiogenesis (VEGF, erythropoietin). HIF is a dimeric transcription factor comprising a regulatory a subunit and constitutively expressed β subunit. HIF-α availability is controlled at the level of protein stability and synthesis by changes in oxygen concentration and growth factors, respectively. Over-expression of HIF-α occurs in most human cancers due to changes in micro-environmental stimuli (e.g. hypoxia, growth factors) and genetic abnormalities that lead to loss of tumour suppressor function (e.g. p53, PTEN, VHL) or oncogenic activation (e.g. Ras, Myc, Src). Thus, targeting HIF function in cancer is an attractive strategy for the development of new anti-cancer agents.

The inventors have developed a cell-based reporter screen (known as “U2OS-HRE-luc”) that was used to identify novel small molecule inhibitors of HIF activity. Using this assay, they have now found that one of their hit compounds (which is referred to herein as the compound represented by formula I or simply “formula I” or “HIF-Inhib1”) inhibits both HIF activity and HIF-α expression in response to hypoxia and growth factors in several cancer cell lines. As such, they are the first group to have demonstrated a therapeutic use for the lead compound, which can be used in the treatment or prevention of cancer. In addition, the inventors propose that compound of formula (I) also has use in other settings where blockade of HIF is therapeutically beneficial (e.g. in the hepatitis C viral (HCV) infection life cycle and hepatoma cell migration).

Hence, in a first aspect of the invention, there is provided a compound of formula (I):—

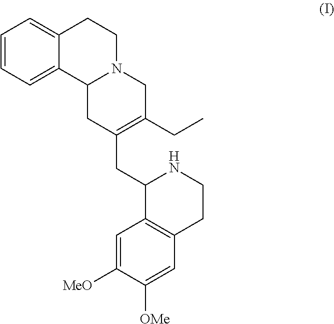 [SMILES: CCC1=C(CC2NCCc3cc(OC)c(OC)cc32)CC2c3ccccc3CCN2C1]

or a functional analogue, or derivative, or pharmaceutically acceptable salt or solvate thereof, for use in therapy or as a medicament.

In a second aspect, there is provided a compound of formula (I), or a functional analogue, or derivative, or pharmaceutically acceptable salt or solvate thereof, for use in treating, preventing or ameliorating a disease characterised by abnormal levels of hypoxia-inducible factor (HIF) activity, preferably cancer.

In a third aspect, there is provided a method of treating, preventing or ameliorating a disease characterised by abnormal levels of hypoxia-inducible factor (HIF) activity, preferably cancer, the method comprising administering, to a subject in need of such treatment, a therapeutically effective amount of a compound of formula (I), or a functional analogue, or derivative, or pharmaceutically acceptable salt or solvate thereof.

Advantageously, the inventors have shown that the compound of formula (I) not only effectively inhibits HIF activity, but also HIF-α expression in response to hypoxia and growth factors in several cancer cell lines. In addition, they have also found that compound (I) inhibits the growth of a panel of tumour cell lines at submicromolar concentrations. Evaluation of compound (I) showed that it has favourable pharmacokinetic properties in vivo and mice could tolerate a maximum dose of up to 100 mg/kg daily dosing by intraperitoneal (IP) injection. Based on these promising initial studies, the inventors went on to investigate the effects of compound (I) on growth of PC3 prostate carcinoma cells grown orthotopically. Surprisingly, the inventors found that compound (I) significantly blocked tumour growth and the incidence of metastasis at local and distant lymph nodes. In addition, compound (I) also blocked HIF-1α and VEGF expression in the orthotopic PC3 prostate carcinoma model.

Along with significant findings, the inventors also went on to identify a potential mechanism of action for compound (I) in that it affects key components of the translational machinery that control HIF-α protein synthesis. Compound (I) is structurally similar to emetine, a known protein synthesis inhibitor. However, surprisingly and advantageously, the inventors have found that compound (I) is at least 100-fold less toxic than emetine on tumour cells. Interestingly, previous studies have shown that emetine targets the 40S ribosome at the level of the ribosomal protein S14. Since phosphorylation of the eukaryotic initiation factor eIF-2α regulates translation initiation from the 40S ribosome, the inventors next assessed eIF-2α phosphorylation in response to compound (I). Their initial studies have shown that both emetine and compound of formula (I) block eIF-2α phosphorylation, suggesting that they may have a similar target profile.

Hence, preferably the compound of formula (I), or a functional analogue, or derivative, or pharmaceutically acceptable salt or solvate thereof, inhibits the hypoxia-inducible factor (HIF) transcriptional complex, i.e. it is a HIF pathway inhibitor. The inventors observed that the compound's IC₅₀ for inhibiting HIF activity in the U2OS-HRE-luc cell-based assay that was used is in the submicromolar range, i.e. ˜0.5 μM.

More preferably, the compound of formula (I), or a functional analogue, or derivative, or pharmaceutically acceptable salt or solvate thereof, reduces or blocks expression of hypoxia-inducible factor-1 alpha (HIF-1α). The inventors found that the compound's potency in blocking HIF-1α protein induction in hypoxia directly correlates with its IC₅₀ for inhibiting HIF activity, i.e. in the range of about 0.25-0.5 μM.

Preferably, the compound of formula (I), or a functional analogue, or derivative, or pharmaceutically acceptable salt or solvate thereof, reduces or blocks expression of vascular endothelial growth factor (VEGF). The compound's potency in blocking VEGF induction in hypoxia directly correlates with its IC₅₀ for inhibiting HIF activity, i.e. ˜0.25-0.5 μM.

Preferably, the compound of formula (I), or a functional analogue, or derivative, or pharmaceutically acceptable salt or solvate thereof, reduces or blocks eIF-2α phosphorylation. The compound's potency in blocking eIF-2α phosphorylation directly correlates with its IC₅₀ for inhibiting HIF activity, i.e. ˜0.25-0.5 μM.

The inventors believe that compound of formula (I), or a functional analogue, or derivative, or pharmaceutically acceptable salt or solvate thereof, can be used to treat any disease resulting from abnormal levels of HIF or HIF activity. In one embodiment, abnormal HIF levels may be decreased with respect to those in a healthy individual. However, preferably the disease is characterised by elevated HIF activity with respect to a healthy individual. In some embodiments, in such diseases, HIF is constitutively upregulated and HIF-α (HIF-1α or HIF-2α) protein is overexpressed. For example, the hepatitis C viral (HCV) infection life cycle is known to result in elevated HIF activity, and so hepatitis C can be treated using the compound of formula (I), or a functional analogue, pharmaceutically acceptable salt or solvate thereof.

Compound of formula (I), or a functional analogue, or derivative, or pharmaceutically acceptable salt or solvate thereof, can be used to treat any tumour or cancer-based disease where HIF is constitutively upregulated and HIF-α (HIF-1α or HIF-2α) protein is overexpressed. For example, the cancer may be a solid tumour or solid cancer. Preferably, compound of formula (I), or a functional analogue, or derivative, or pharmaceutically acceptable salt or solvate thereof, is used to treat prostate cancer. Hepatoma cell migration may also be treated.

The skilled person will appreciate that although compound of formula (I) has been demonstrated in the Examples as showing surprising efficacy for inhibiting HIF and therefore exhibits utility for treating tumours and cancers, various functional analogues of compound (I) can also be used, as they can also inhibit HIF. A functional analogue can be defined as being any compound which exhibits at least 80% HIF inhibition compared to compound (I) using the U2OS-HRE-luc cell-based assay without affecting cell viability, i.e. the analogue is not toxic. Toxicity can be defined as being more than 20% cell death within 24 hours, and so functional analogues should not cause more than 20% death.

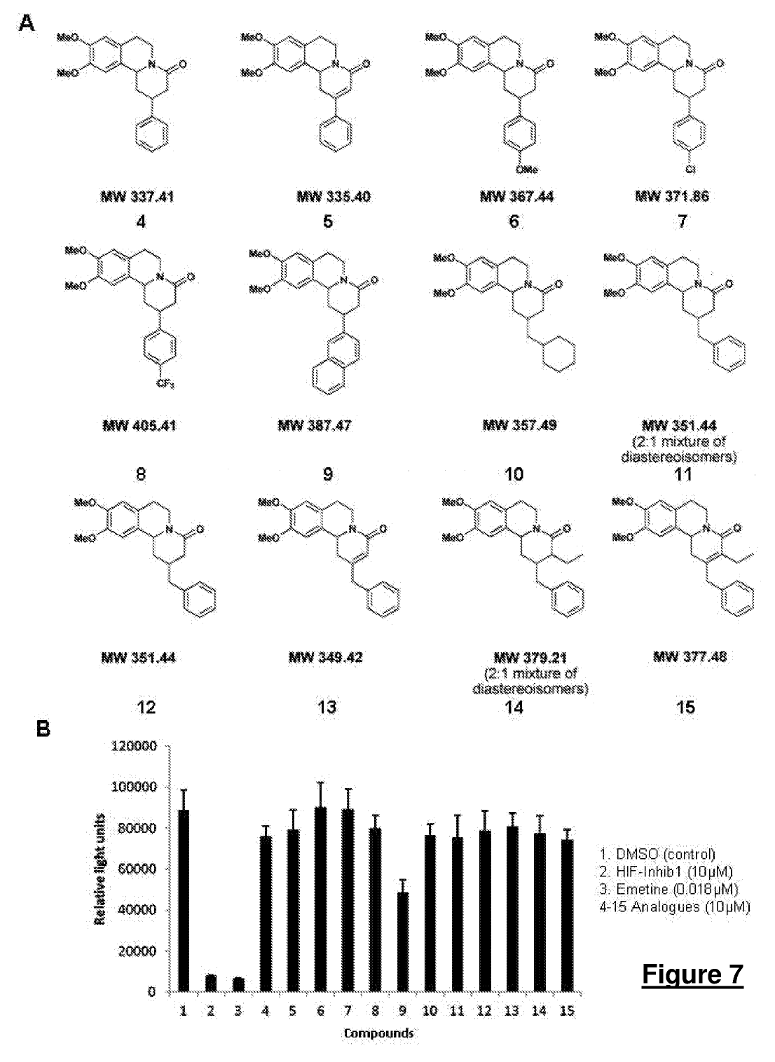

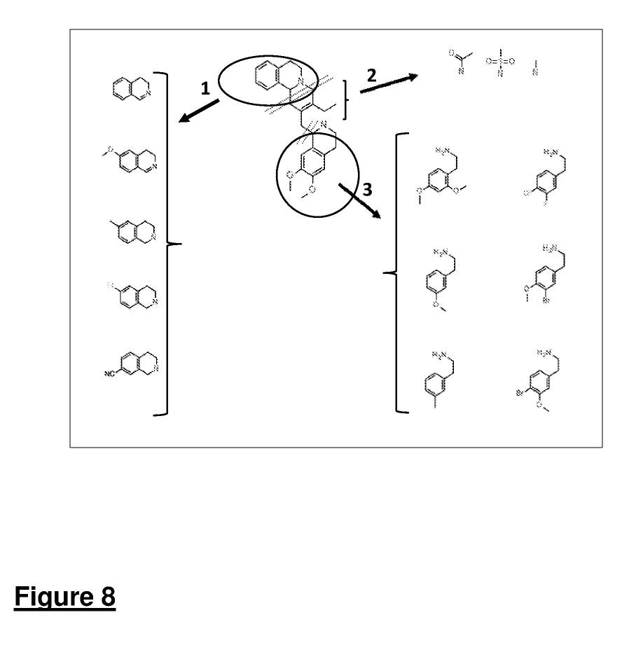

The inventors have investigated several analogues of compound (I), which are shown in FIGS. 7-12. For example, the chemical structure of compound (I) can be broken down into three subunits as shown by the double lines in the centre of FIG. 8. Arrows 1 and 3 in FIG. 8 indicate that there are up to 6-11 independent chemical groups in combination with up to three separate cores resulting in a variety of functional analogues. Accordingly, preferred analogues of compound (I) are shown in FIG. 8.

Compound (I), for use, in the invention, may be chiral. Hence, the compound (I) may include any diastereomer and enantiomer of the formula represented by (I). Diastereomers or enantiomers of (I) are believed to display potent HIF inhibitory activity, and such activities may be determined by use of appropriate in vitro and in vivo assays, which will be known to the skilled technician. Compounds defined by formula (I) can therefore include analogues as racemates. Alternatively, the compounds of formula (I) can be pairs of diastereoisomers, or individual enantiomers, including the threo- and erythro-pair of diastereoisomers and the individual threo and erythro enantiomers.

Preferably, the compound (I) is the S, R enantiomer, i.e. (S)-2-(((R)-6,7-dimethoxy-1,2,3,4-tetrahydroisoquinolin-1-yl)methyl)-3-ethyl-1,6,7,11b-tetrahydro-4H-pyrido[2,1-a]isoquinoline.

It will also be appreciated that compounds for use in the invention may also include pharmaceutically active salts, e.g. the hydrochloride.

The inventors have realised that the compound of formula (I) is a surprisingly effective HIF pathway inhibitor.

Hence, in a fourth aspect, there is provided a hypoxia-inducible factor (HIF) pathway inhibitor comprising a compound of formula (I), or a functional analogue, or derivative, or pharmaceutically acceptable salt or solvate thereof.

In a fifth aspect, there is provided a compound of formula (I), or a functional analogue, or derivative, or pharmaceutically acceptable salt or solvate thereof, for use as hypoxia-inducible factor (HIF) pathway inhibitor.

It will be appreciated that the compound of formula (I), or a functional analogue, or derivative, or pharmaceutically acceptable salt or solvate thereof according to the invention may be used in a medicament which may be used in a monotherapy (i.e. use of compound (I) alone), for treating, ameliorating, or preventing a disease characterised by abnormal levels of hypoxia-inducible factor (HIF) activity, preferably cancer. Alternatively, the compound of formula (I), or a functional analogue, or derivative, or pharmaceutically acceptable salt or solvate thereof according to the invention may be used as an adjunct to, or in combination with, known therapies for treating, ameliorating, or preventing cancer.

The compound of formula (I), or a functional analogue, or derivative, or pharmaceutically acceptable salt or solvate thereof according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising the compound of formula (I), or a functional analogue, or derivative, or pharmaceutically acceptable salt or solvate thereof according to the invention may be used in a number of ways. For instance, oral administration may be required, in which case the compound may be contained within a composition that may, for example, be ingested orally in the form of a tablet, capsule or liquid. Compositions comprising the compounds of the invention may be administered by inhalation (e.g. intranasally). Compositions may also be formulated for topical use. For instance, creams or ointments may be applied to the skin.

Compounds according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with compounds used according to the invention is required and which would normally require frequent administration (e.g. at least daily injection).

In a preferred embodiment, compounds and compositions according to the invention may be administered to a subject by injection into the blood stream or directly into a site requiring treatment. Injections may be intravenous (bolus or infusion) or subcutaneous (bolus or infusion), or intradermal (bolus or infusion).

It will be appreciated that the amount of the compound that is required is determined by its biological activity and bioavailability, which in turn depends on the mode of administration, the physiochemical properties of the compound, and whether it is being used as a monotherapy, or in a combined therapy. The frequency of administration will also be influenced by the half-life of the compound within the subject being treated. Optimal dosages to be administered may be determined by those skilled in the art, and will vary with the particular compound in use, the strength of the pharmaceutical composition, the mode of administration, and the advancement of the cancer. Additional factors depending on the particular subject being treated will result in a need to adjust dosages, including subject age, weight, gender, diet, and time of administration.

Generally, in one embodiment, a daily dose of between 0.01 μg/kg and 500 mg/kg of body weight, or between 0.1 mg/kg and 200 mg/kg body weight of the compound according to the invention may be used for treating, ameliorating, or preventing cancer depending upon which compound or analogue is used. The compound may be administered before, during or after onset of the cancer to be treated. Daily doses may be given as a single administration (e.g. a single daily injection). Alternatively, the cancer may require administration twice or more times during a day. As an example, compound (I) may be administered as two (or more depending upon the severity of the cancer being treated) daily doses of between 25 mg and 7000 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of the compounds according to the invention to a patient without the need to administer repeated doses.

Known procedures, such as those conventionally employed by the pharmaceutical industry (e.g. in vivo experimentation, clinical trials, etc.), may be used to form specific formulations comprising the compounds according to the invention and precise therapeutic regimes (such as daily doses of the compounds and the frequency of administration). The inventors believe that they are the first to describe a pharmaceutical composition for treating cancer, based on the use of the compounds of the invention.

Hence, in a sixth aspect of the invention, there is provided a pharmaceutical composition comprising a compound of formula (I), or a functional analogue, or derivative, or pharmaceutically acceptable salt or solvate thereof, and a pharmaceutically acceptable vehicle.

The pharmaceutical composition can be used in the therapeutic amelioration, prevention or treatment in a subject of a disease characterised by abnormal levels of hypoxia-inducible factor (HIF) activity, preferably cancer. Thus, the composition is preferably an anti-cancer pharmaceutical composition.

Preferably, the compound (I) is (S)-2-(((R)-6,7-dimethoxy-1,2,3,4-tetrahydroisoquinolin-1-yl)methyl)-3-ethyl-1,6,7,11b-tetrahydro-4H-pyrido[2,1-a]isoquinoline.

The invention also provides in a seventh aspect, a process for making the composition according to the sixth aspect, the process comprising contacting a therapeutically effective amount of a compound of formula (I), or a functional analogue, pharmaceutically acceptable salt or solvate thereof, and a pharmaceutically acceptable vehicle.

A “subject” may be a vertebrate, mammal, or domestic animal. Hence, compounds, compositions and medicaments according to the invention may be used to treat any mammal, for example livestock (e.g. a horse), pets, or may be used in other veterinary applications. Most preferably, however, the subject is a human being.

A “therapeutically effective amount” of compound is any amount which, when administered to a subject, is the amount of drug that is needed to treat the target disease, or produce the desired effect, i.e. inhibits HIF activity. For example, the therapeutically effective amount of compound used may be from about 0.01 mg to about 800 mg, and preferably from about 0.01 mg to about 500 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. However, the pharmaceutical vehicle may be a liquid, and the pharmaceutical composition is in the form of a solution. Liquid pharmaceutical compositions, which are sterile solutions or suspensions, can be utilized by, for example, intramuscular, intrathecal, epidural, intraperitoneal, intravenous and particularly subcutaneous injection.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

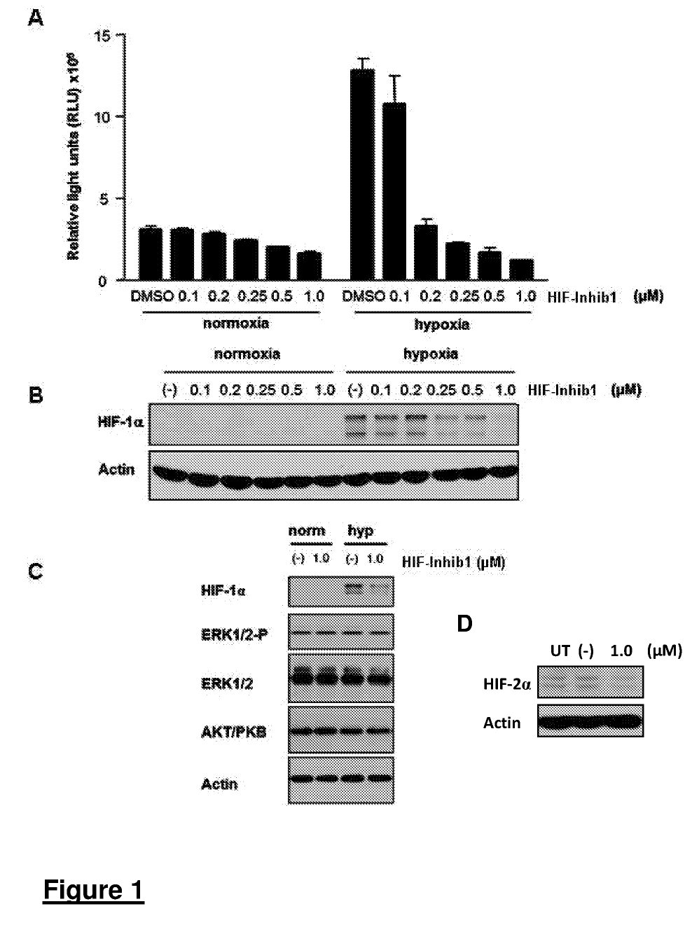

FIG. 1 shows that compound I of the invention (referred to herein as “HIF-Inhib1”) blocks HIF activity and HIF-α protein induction in hypoxia in a dose-dependent manner without affecting HIF-1β or key cellular signaling proteins, ERK1/2 and AKt/PKB. FIG. 1A: Graph shows HIF (HRE-luciferase) activity measured as relative light units (RLU) in U2OS-HRE-luc cells in response to HIF-Inhib1 treatment over a dose range as indicated in normoxia or hypoxia for 16 hours. U2OS-HRE-luc described in A, were harvested for western blot analysis. FIG. 1B: Western blots show the effects of HIF-Inhib1 on HIF-1α protein in normoxia or hypoxia. Actin was used as a load control. FIG. 1C: Western blots show HIF-1α, phosphorylated ERK1/2 (ERK1/2-P), and AKT/PKB proteins in the absence (−) and presence of 1 μM HIF-Inhib1 in normoxia (norm) or hypoxia (hyp). Actin was used as a load control. FIG. 1D: Western blots show the effects of HIF-Inhib1 (1 μM) on HIF-2α protein levels. UT (untreated), and DMSO treated (−) controls are indicated;

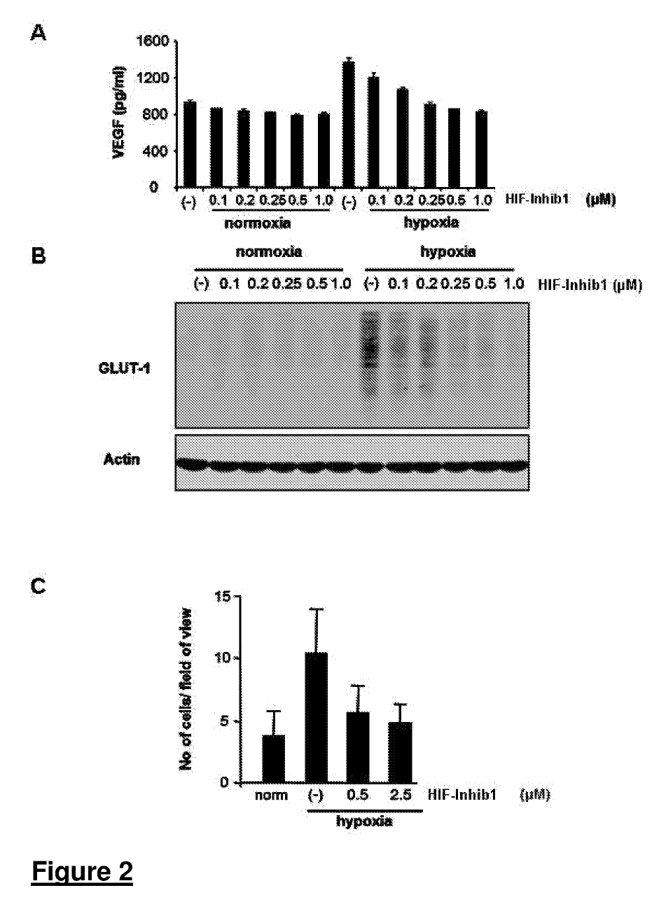

FIG. 2 shows that HIF-Inhib1 blocks the induction of HIF targets (GLUT1 and VEGF) and tumour cell migration in hypoxia. FIG. 2A: Graph shows vascular endothelial growth factor (VEGF) protein expression measured by ELISA in U2OS-HRE-luc cells in response to HIF-Inhib1 treatment over a dose range as indicated in normoxia or hypoxia for 16 hours, FIG. 2B: U2OS-HRE-luc described in A, were harvested for western blot analysis. Western blots show the effects of HIF-Inhib1 on glucose transporter1 (GLUT1) protein induction in normoxia or hypoxia. Actin was used as a load control. FIG. 2C: Graph shows tumour cell migration (number (no) of migrated cells/field of view) in the absence (−) and presence of 0.5 or 2.5 μM HIF-Inib1 in normoxia (norm) or hypoxia (hyp) for 16 hours;

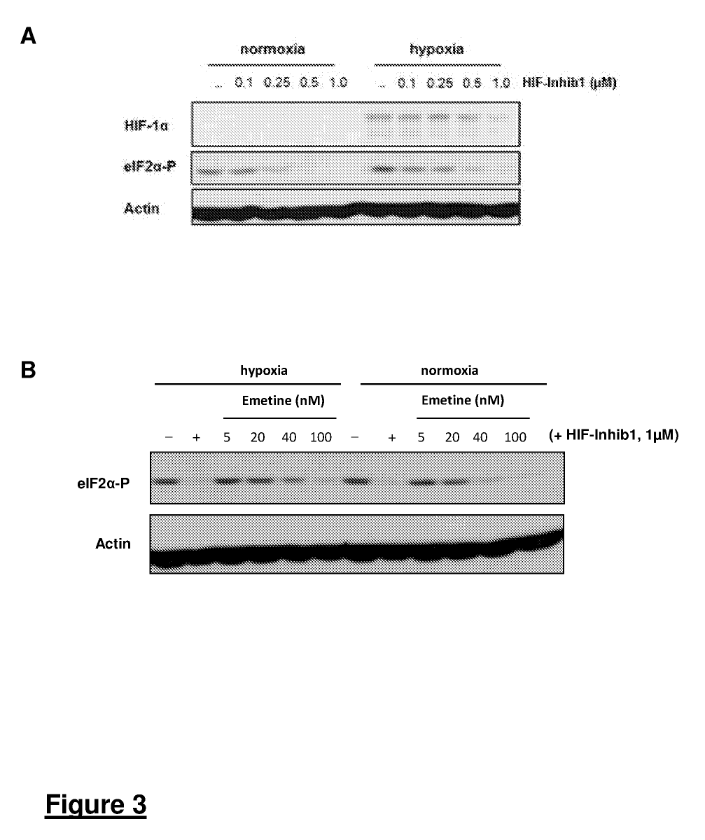

FIG. 3 shows that HIF-Inhib1 targets the protein translation machinery. FIG. 3A: Western blots show the effects of HIF-Inhib1 treatment over a dose range on HIF-1α and phosphoryated eIF-2α (eIF-2α-P) proteins in U2OS-HRE-luc cells in normoxia or hypoxia for 16 hours. Actin was used as a load control. FIG. 3B: Western blots show the effects of HIF-Inhib1 (1 μM) or emetine treatment over a dose range on phosphorylated eIF-2α (eIF-2α-P) in U2OS-HRE-luc cells in normoxia or hypoxia for 16 hours. Actin was used as a load control;

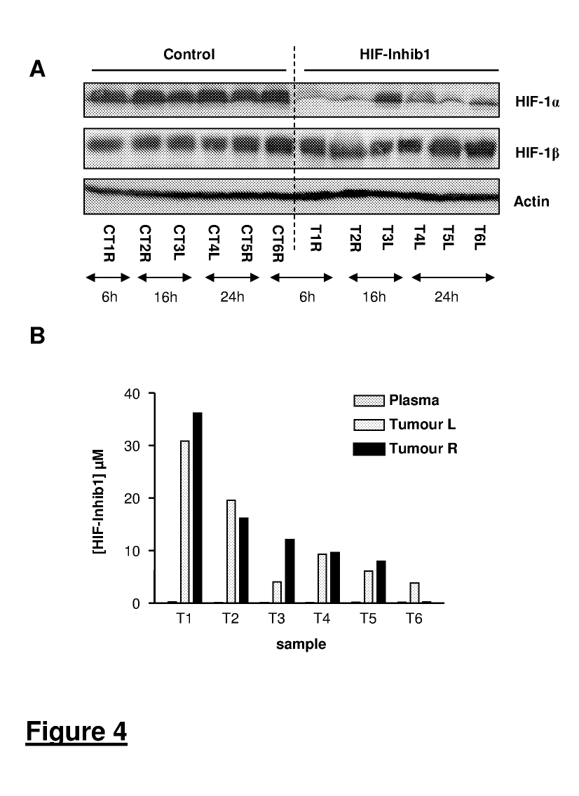

FIG. 4 shows pharmacodynamic (PD) and pharmacokinetic (PK) effects of 100 mg/kg daily dosing by intraperitoneal (IP) injection of HIF-Inhib1 in a human PC3LN5 subcutaneous mouse xenograft model. FIG. 4A: Western blots show the effects of control (CT) or HIF-Inhib1 treatment (T) on HIF-1α protein levels as a PD endpoint in PC3 tumour xenografts excised from left (L) or right (R) subcutaneous hindlimbs. Actin was used as a load control. FIG. 4B: Graph shows levels of HIF-Inhib1 (μM) in the tumours described in A, measured by LC/MS analyses;

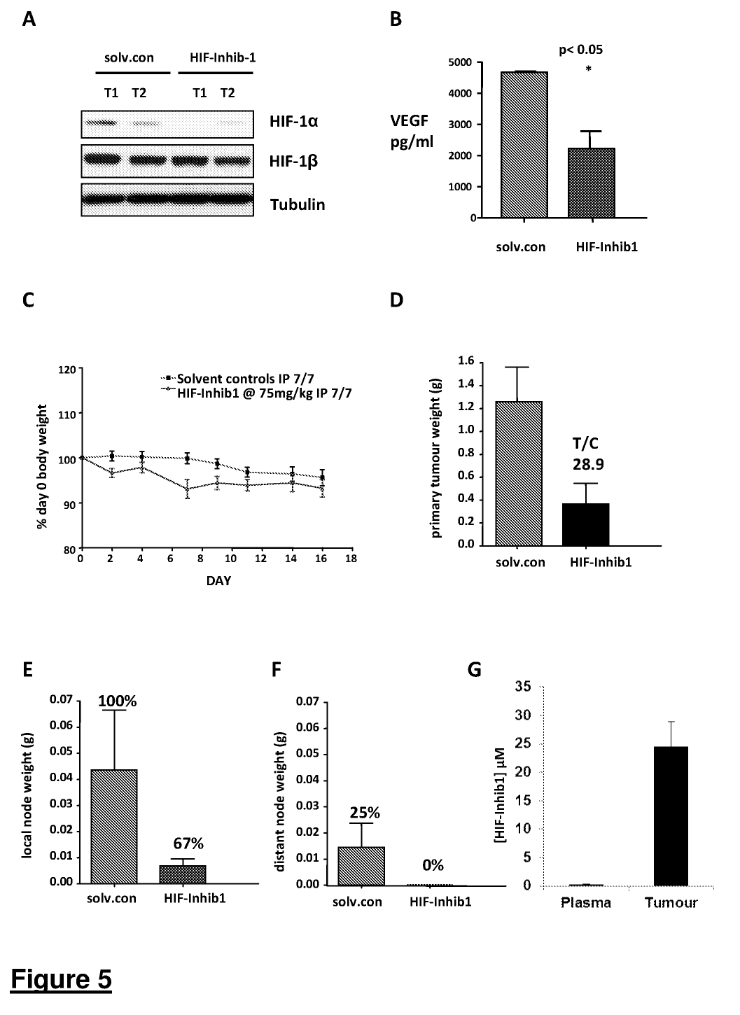

FIG. 5 shows that HIF-Inhib1 blocks HIF-α, VEGF, tumour growth and metastasis (local and distant) in a human PC3 orthotopic mouse xenograft model. FIG. 5A: Western blots shows the effects of control (solv.con) or HIF-Inhib1 treatment on the levels of HIF-1α and HIF-1β proteins PC3 tumour xenografts excised as indicated at day 16 after 75 mg/kg daily dosing by intraperitoneal injection. FIG. 5B: Graph shows VEGF protein levels (pg/ml) from pooled tumour xenographs described in A. FIG. 5C-F: Graphs show body weight in tumour bearing mice described in A (C), primary tumour weight in grams (g) at day 16 (D), and the weight (g) of local (E) and distant (F) lymph node metastasis. FIG. 5G: Graph shows levels of HIF-Inhib1 (μM) in plasma and pooled tumours described in A, measured by LCMS analyses;

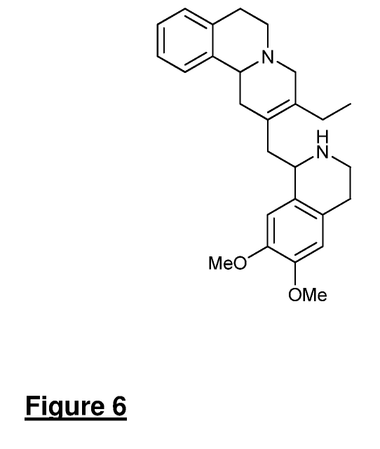

FIG. 6 shows the chemical structure of HIF-Inhib1 according to the invention;

FIG. 7 shows the effects of a series of HIF-Inhib1 analogues on HIF activity in U2OS-HRE-luc cells. FIG. 7A: Structures and molecular weights (MW) are shown for a series of chemical analogues (labelled 4-15) of HIF-Inhib1. FIG. 7B: Graph shows the effects of DMSO control (1), HIF-Inhib1 at 10 μM (2), Emetine at 0.018 μM (3) and analogues (4-15 at 10 μM) on HIF activity (relative light units) in the U2OS-HRE-luc cell-based assay in hypoxia (1% O₂, for 16 hours);

FIG. 8 shows the structures of a panel of functional analogues of HIF-Inhib1 that include a variety of different chemical groups as indicated at positions 1, 2 and 3 within the active phamacophore;

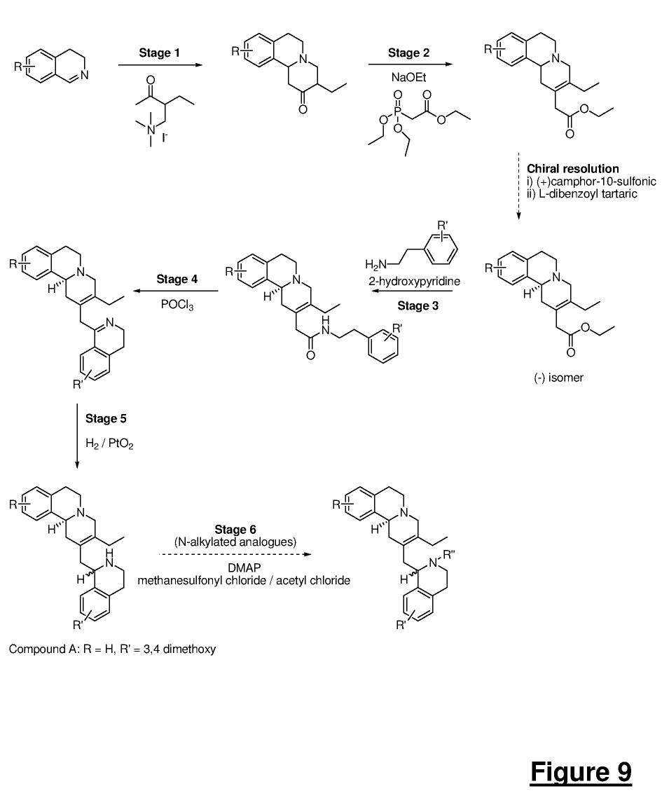

FIG. 9 shows the reaction scheme for synthesising chemical enantiomers and analogues of HIF-Inhib1;

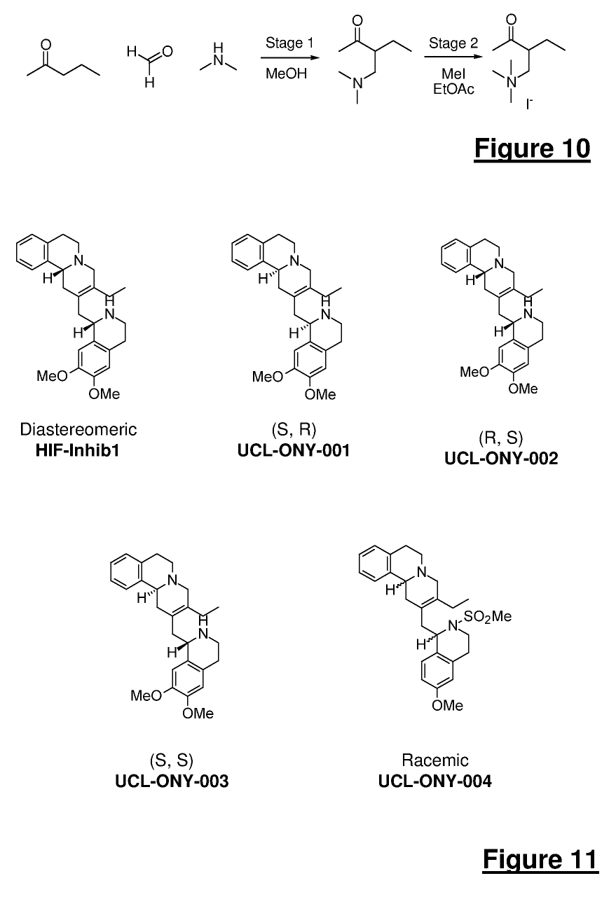

FIG. 10 shows the reaction scheme for synthesising 3-dimethylaminomethyl-pentan-2-one methiodide;

FIG. 11 shows the purified structures of five of the chemical enantiomers and analogues of HIF-Inhib1 which were obtained by using the reaction scheme shown in FIG. 9; and

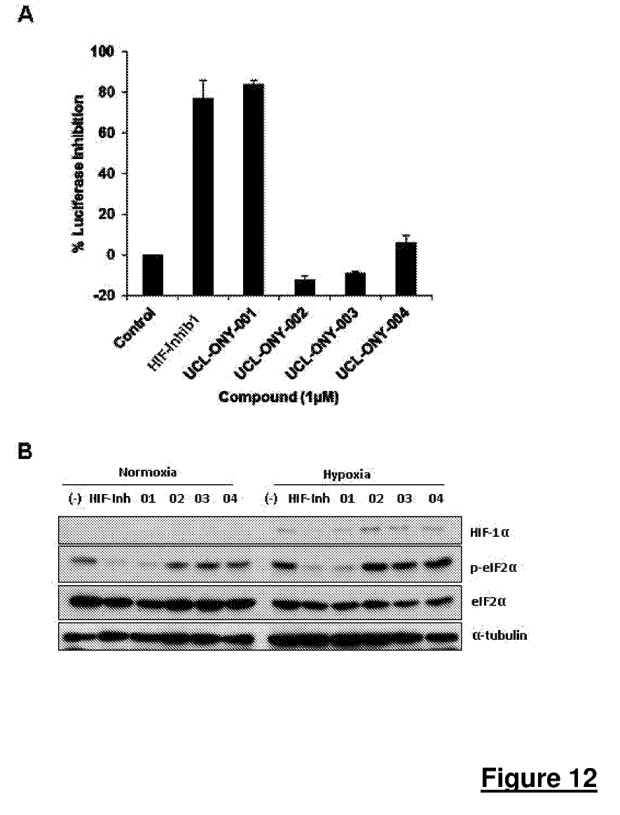

FIG. 12 shows the effects of the chemical enantiomers and analogues shown in FIG. 11 on HIF activity. FIG. 12A is a graph showing the percentage inhibition of luciferase activity in U2OS-HRE cells treated with the compounds shown in FIG. 11. The compounds were dosed at 1 μM and incubated in 1% O2 for 16 hours. FIG. 12B shows western blot analysis of U2OS-HRE cells treated with compounds indicated including HIF-Inhib1 (HIF-Inh) as in FIG. 1C to show inhibitory effects on HIF-1α, phosphorylated and total eIF2α protein levels. Tubulin was used as a loading control. All data shown has been either averaged or is representative of 3 independent experiments.

### EXAMPLES

The inventors have found that the compound represented as formula I, which is shown in FIG. 6, inhibits both HIF activity and HIF-α expression in response to hypoxia and growth factors in several cancer cell lines. Accordingly, compound (I) can be used therapeutically for the treatment of solid tumours. The compound represented by formula I is known herein as “HIF-Inhib1”.

### Example 1—Compound I of the Invention Blocks HIF-1α Protein Induction in Hypoxia

U2OS-HRE-luc cells were exposed to normoxia or hypoxia (1% O₂) for 16 hours in the presence of DMSO (control) or HIF-Inhib1 over a concentration range (0.1-1 μM). Cells were harvested and assessed for HRE-luciferase activity as a measure of HIF activity, and for western blot analysis.

### Results

As shown in FIG. 1, compound I of the invention blocks HIF activity (HRE-luciferase activity measured as relative light units, RLU) in a dose dependent manner (FIG. 1A). This dose-dependent inhibitory effect on HIF activity was found to directly correlate with blockade of HIF-α protein induction in hypoxia (FIG. 1B). The inventors found that compound I of the invention had no significant effect on the expression of key cellular signaling proteins, ERK1/2 and AKt/PKB (FIG. 1C) at doses which significantly affected HIF, indicating a specific inhibitory effect of compound I of the invention on the HIF pathway. In addition, the inventors show that compound 1 blocks HIF-2α protein induction in hypoxia.

### Example 2—Compound I Blocks HIF Targets (GLUT1 and VEGF) and Tumour Cell Migration in Hypoxia

U2OS-HRE-luc cells were exposed to normoxia or hypoxia (1% O₂) for 16 hours in the presence of DMSO (control) or HIF-Inhib1 over a concentration range (0.1-1 μM). Cells were harvested and assessed for VEGF and GLUT1 protein levels using a quantitative ELISA or by western blot analysis respectively. In addition, tumour cells were exposed to 0.5 or 2.5 μM HIF-Inhib1 in hypoxia, and tumour cell migration was measured using a 2-dimensional filter-based migration assay.

### Results

FIG. 2 shows that compound I of the invention blocks the induction of HIF target proteins, VEGF and GLUT1 (FIG. 2A-B) in a dose-dependent manner. These data correlate directly with the dose-dependent inhibitory effects HIF-Inhib1 on HIF activity and HIF-1α protein in hypoxia shown in FIG. 1A-1B. In addition, FIG. 2C shows that HIF-Inhib1 also reduces tumour cell migration induced in hypoxia in a dose-dependent manner, and is consistent with blockade of the HIF pathway.

### Example 3—Compound I Targets Key Components of the Protein Translation Machinery

U2OS-HRE-luc cells were exposed to normoxia or hypoxia (1% O₂) for 16 hours in the presence of DMSO (control) or HIF-Inhib1 over a concentration range (0.1-1 μM). Cells were harvested and components of the protein translational machinery were assessed by western blot analysis.

### Results

FIG. 3 shows that compound I targets components of the protein translation machinery. The inventors found that HIF-Inhib1 blocked eIF-2α phosphorylation in a dose-dependent manner, indicating that compound I affects protein translation. These data correlate directly with the dose-dependent inhibitory effects HIF-Inhib1 on HIF activity and HIF-1α protein. Furthermore, the inventors have found that emetine, a known protein translation inhibitor, and analogue of compound 1 also blocks eIF-2α phosphorylation.

### Example 4—Compound I of the Invention Blocks HIF-1α and Shows Good Bioavailability In Vivo

HIF-Inhib1 was administered IP dose of 100 mg·kg⁻¹ to Nu mice with PC3LN5 xenografts. Mice were killed at 24 h and xenografts removed for PD/PK analysis. Tumour samples were homogenised with 3× (v/w) PBS and 50 μL extracted by addition of 150 μL of methanol. Tumour extracts were analysed by LCMS using reverse-phase Synergi Polar-RP (Phenomenx, 50×2.1 mm) analytical column and positive ion mode ESI+ MRM.

### Results

FIG. 4 shows that the concentrations of compound I between left and right flank subcutaneous tumours were comparable. Tumour concentrations ranged between 1.9-35 μM. Plasma concentrations ranged between 0.07 and 0.3 μM.

### Example 5—Compound I Blocks HIF-α, VEGF, Tumour Growth and Metastasis (Local and Distant) in a Human PC3LN5 Orthotopic Mouse Xenograft Model

PC3LN5 (10⁵ cells) were implanted intraprostatically into mice (Nu) and tumours were allowed to develop for 12 days. Mice received HIF-Inhib1 (75 mg·kg⁻¹) by IP injection daily for 2.5 weeks. Plasma and tumour samples were taken 24 h after the last dose and analysed by LCMS. Tumours were excised and homogenised, and assessed for PD endpoints HIF-1α and VEGF proteins. Local and distant lymph node metastases were also evaluated.

### Results

FIG. 5 shows that compound I blocks HIF-1α and VEGF protein in PC3LN5 orthotopic tumours in vivo. Mouse body weight was not significantly affected over 16 days of daily dosing with HIF-Inhib1, indicating minimal toxicity. HIF-Inhib1 significantly blocked tumour growth and metastasis (local and distant) in the PC3LN5 orthotopic xenograft model. HIF-Inhib1 showed a good PK profile in tumours, indicating good bioavailability to the tumour.

### Example 6—Chemical Structure of Compound I of the Invention

Referring to FIG. 6, there is shown the structure of compound (I), i.e. HIF-Inhib1.

### Example 7—Analogues of Compound I (Batch 1)

A series of analogues (labelled 4-15) of compound I were synthesised, and their structures are shown in FIG. 7. U2OS-HRE-luc cells were exposed to hypoxia (1% O₂) for 16 hours in the presence of DMSO (control), HIF-Inhib1 (10 μM), emetine (0.017 uM) as positive control, and then each of the analogues (4)-(15) as shown in FIG. 7. Cells were harvested and luciferase activity was measured in cell lysates using a standard luminometer. Data was represented as relative light units (RLU) for each condition.

### Results

FIG. 7B shows the effects of the compounds on U2OS-HRE luciferase assay, as a measure of HIF activity. HIF-Inhib1 and emetine significantly blocked HIF activity in hypoxia, while the analogues tested had minimal inhibitory effects.

Referring to FIG. 8, there is shown various other analogues that have been generated, and which show HIF inhibition activity. The chemical structure of compound (I) was broken down into three subunits as shown by the double lines in the centre of the Figure. Arrows 1 and 3 in FIG. 8 indicate that there are up to 11 independent chemical groups in combination with up to 3 separate cores (arrow 2) resulting in a variety of functional analogues.

### Example 8—Analogues of Compound I (Batch 2)

A further series of enantiomers and analogues of compound I were synthesised, and their structures are shown in FIG. 11. The compounds were synthesised using a six step process, as illustrated in the reaction scheme shown in FIG. 9, and explained below. It will be noted that stage 1 of the reaction scheme shown in FIG. 9 requires 3-dimethylaminomethyl-pentan-2-one methiodide, which was itself prepared according to the reaction scheme shown in FIG. 10.

### SYNTHESIS REFERENCES:—

1. Whittaker N.; Openshaw H. T.; Manufacture of 1, 2, 3, 4, 6, 7-hexahydro-2-oxo-11bh-benzo(a)quinolizines; U.S. Pat. No. 3,375,254 A.
2. Whittaker N.; The synthesis of emetine and related compounds. Part IX. The use of Wittig-type reagents in the synthesis of 2,3-dehydroemetine; J. Chem. Soc. C, 1969, 94-100.
Brossi A.; Baumann M.; Chopard-dit-Jean L. H.; Würsch, J.; Schneider, F.; Schnider O.; Helvetica Chimica Acta, 1959, 42 (3), 772-788.

### Synthesis of 3-dimethylaminomethyl-pentan-2-one methiodide

### Stage 1—Condensation

A flask was charged with paraformaldehyde (88 g, 2.9 mol), dimethylamine hydrochloride (150 g, 1.8 mol), pentan-2-one (590 mL, 5.5 mol) and methanol (450 mL). The flask was purged with nitrogen and heated at reflux overnight. The solution was cooled and the pH adjusted to 9 with 2M aqueous NaOH. The product was extracted into diethyl ether (3×1400 mL), dried over magnesium sulphate and concentrated in vacuo. The crude mixture was distilled under reduced pressure (vigreux column, 20 torr, head 66-74° C.) to obtain ˜150 mL of a yellow liquid. This was purified by column chromatography on silica (3 kg) eluting with 1% 7N MeOH in DCM and then 2% 7N MeOH in DCM to obtain 58 g of product as a yellow oil (22% yield).

### Stage 2—Salt Formation

The amine (55 g, 0.4 mol) was filtered under a blanket of nitrogen (to remove oxidation products from storage) into a flask fitted with an overhead stirrer. Ethyl acetate (250 mL) was added and the mixture stirred at RT under nitrogen. Methyl iodide (109 g, 0.8 mol) was then added over 5 minutes with cooling provided to maintain T<30° C. The mixture was stirred overnight at RT and then filtered under a blanket of nitrogen washing with ethyl acetate (300 mL). The precipitate was pulled dry on the filter and oven dried under vacuum at 45° C. to obtain 99 g of a white solid (91% yield).

### Synthesis of Compounds Shown in FIG. 11

### Stage 1—Cyclisation

A flask was charged with 3-dimethylaminomethyl-pentan-2-one methiodide (58 g, 206 mmol) and dihydroisoquinoline (9 g, 69 mmol) and suspended in ethanol (225 mL). The mixture was heated to reflux under nitrogen overnight. The mixture was cooled to room temperature and filtered. The filter was washed with ethanol (50 mL) and the filtrate combined and concentrated in vacuo to yield a yellow oil (24 g). This was purified by column chromatography on silica (500 g) eluting with 1% ethyl acetate in heptane followed by 20% and 30% ethyl acetate in heptane. The product fractions were combined and the solvent removed in vacuo. The resulting yellow solid was further purified by slurry in ethanol (60 mL) to yield a white solid (7.7 g, 49%). A further crop of product (2.2 g, 14%) was obtained upon concentration of the ethanolic washings to half volume.

### Stage 2—Horner Wadsworth Emmons Reaction

A flask was charged with diethyl phthalate (6.9 g, 31 mmol), sodium ethoxide solution (50.3 g, 155 mmol, 21% wt in ethanol), and ethanol (90 mL) and cooled to −5° C. under an atmosphere of nitrogen. Triethyl phosphonoacetate (10.9 g, 49 mmol) was added dropwise maintaining a temperature <5° C. The solution was allowed to warm to 10° C. and stirred for 1 hr before being cooled to 0° C. Stage 1 (8.9 g, 39 mmol) was added in one portion and the mixture stirred for 3 hrs at RT followed by 2 hours at reflux. The ethanol was removed in vacuo and the residue partitioned between toluene (400 mL) and water (400 mL). The phases were separated and the aqueous extracted with a further portion of toluene (50 mL). The combined organics were extracted into 1M HCl (500 mL) which was then basified with NaOH and twice extracted into diethyl ether (2×400 mL). The organics were dried over MgSO4, filtered and concentrated to yield a light yellow oil (11.4 g, 98%). The oil was purified by silica chromatography (225 g Si) eluting with 15% ethyl acetate in heptane followed by 30% ethyl acetate in heptane to yield the product as an oil (9.6 g, 82%).

### Chiral Resolution

### (+)-Camphor-Sulfonic Acid

A flask was charged with stage 2 (4.2 g, 14 mmol) and TBME (42 mL) and stirred at 40° C. A solution of (1S)(+)Camphor-10-sulfonic acid (3.2 g, 14 mmol) in warm ethanol (14 mL) was then added in one portion and the solution stirred at RT for 3 hrs. The camphor-sulfonic acid salt was then collected by filtration, washing with TBME (50 mL), and oven dried under vacuum at 40° C. (3.4 g, 91% recovery, 99.4% ee). Freebasing this salt by partition with 1M NaOH (100 mL) and TBME (100 mL) yielded the (+) enantiomer of stage 2.

The liquors from the crystallisation were concentrated in vacuo and partitioned between 1M NaOH (80 mL) and TBME (80 mL). The organics were dried over MgSO4 and concentrated in vacuo to yield the freebase as an oil (2.4 g, 85% (−), 15% (+)).

### (−)-Diparatoluoyl-Tartaric Acid

A flask was charged with the residue from the first crystallisation (2.4 g, 8 mmol) and TBME (48 mL) and stirred at 40° C. A solution of diparatoluoyl-L-tartaric acid (3.1 g, 8 mmol) in warm ethanol (8 mL) was then added in one portion and the mixture stirred at RT overnight. The diparatoluoyl-tartaric acid salt was then collected by filtration, washing with TBME (50 mL). The salt (3.6 g) was then recrystallised from a mixture of hot TBME (36 mL, 10 vol) and ethanol (12 mL, 3.3 vol) and oven dried under vacuum at 40° C. (2.8 g, 60% recovery, 97.9% ee). Freebasing this salt by partition with 1M NaOH (8 mL) and TBME (8 mL) yielded the (−) enantiomer of stage 2.

The liquors from the crystallisation were concentrated in vacuo and partitioned between 1M NaOH (5 mL) and TBME (50 mL). The organics were dried over MgSO4 and concentrated in vacuo to yield the freebase as an oil (1.6 g). This was re-subjected to the procedure above to provide an additional 0.2 g of the (+) enantiomer (99.6% ee) and 0.8 g of the (−) enantiomer (99.3% ee).

### Stage 3—Amide Formation

A flask was charged with stage 2 (2.3 g, 7.6 mmol), 2-hydroxypyridine (0.7 g, 7.6 mmol) and the substituted phenethylamine (11.4 mmol). The mixture was heated at 165° C. for 4 hrs and cooled to RT. Water (40 mL) and diethyl ether (12 mL) were added and the mixture slurried for 30 minutes. The precipitate was collected by filtration and washed with diethyl ether (20 mL) before being oven dried under vacuum at 45° C. to yield a white solid (2.5 g, 76%).

### Step 4—Cyclisation

A flask was charged with stage 4 (2.5 g, 5.6 mmol) plus toluene (45 mL). POCl3 (1.7 g, 11.3 mmol) was added and the mixture heated to 80° C. for 2 hrs. A gum formed on the flask walls that was subsequently taken into solution by the addition of acetonitrile (10 mL). The solution was heated to 80° C. for a further 2 hours and cooled to 50° C. before the addition of methanol (20 mL). The solvents were removed in vacuo and the residue partitioned between 1M NaOH (50 mL) and DCM (50 mL). The organics were dried over Mg-SO4 and evaporated to dryness to give a yellow oil (3 g, assume 100% yield). The crude product plus trimethyl phosphate was used without purification in the following step.

### Step 5—Hydrogenation

A flask was charged with crude stage 4 (5.6 mmol) in methanol (25 mL). 2M HCl (25 mL) was added and the flask purged with nitrogen. Platinum (IV) oxide (64 mg, 0.3 mmol) was added and the flask sparged with hydrogen for 6 hrs before being stirred overnight under a head of hydrogen. The mixture was filtered on Celite and the filtrate concentrated in vacuo to remove methanol. The aqueous was basified with 1% Na2CO3 and the precipitate collected by filtration (˜3 g). The product diastereomers were purified by column chromatography on silica (120 g) eluting with 2% MeOH in DCM then 2% 7N methanolic ammonia in DCM. Clean fractions of the desired stereoisomer (top spot) were combined and evaporated in vacuo to yield an off-white solid (250 mg, 11% yield). Mixed fractions were combined and evaporated in vacuo to give 800 mg of a diastereomer mix enriched in the lower spot (35% yield, ˜0.5:1 mixture). See experiment tables for approximate purities and stereochemical assignment based on literature precedent (Chem. Commun., 2014, 50, 1238).

### Step 6—N-Alkylation

A sealed tube was loaded with stage 5 (0.1 mmol) and DMAP (0.4 mmol) in DCM (1 mL). The appropriate alkylating agent was charged (2 eq) and the tube purged with nitrogen, sealed and stirred overnight at room temperature. The mixture was blown to dryness and partitioned between diethyl ether (1 mL) and 1M NaOH (1 mL). The organic phase was blown to dryness and columned on a 2 g silica cartridge eluting 8×5 mL fractions of 1% MeOH/DCM. Product fractions were combined and evaporated to dryness in vacuo to yield an off white solid (40-60% yield).

The compounds were analysed and the proton NMR assignments were made as set out in Table 1.

TABLE 1
The structure, stereochemistry, LCMS purity and NMR assignments for
enantiomers and analogues of compound I

| Structure | Identifier | Stereochemistry | LCMS Purity | NMR |
|---|---|---|---|---|
| 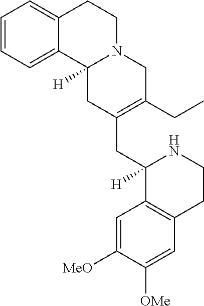 [SMILES: CCC1=C(C[C@H]2NCCc3cc(OC)c(OC)cc32)C[C@H]2c3ccccc3CCN2C1] | UCL-ONY-001 | (S,R) | 82.2% [M + H]⁺ 419.4 | ¹H NMR (MeOD, 270 MHz) δ ppm 1.05 (m, 3H), 2.02-2.35 (m, 3H), 2.44-2.85 (m, 7H), 2.92-3.19 (m, 4H), 3.20-3.29 (m, 1H), 3.32-3.39 (m, 1H), 3.49 (dd, 1H, J = 10.9 Hz, 4.3 Hz), 3.79 (s, 3H), 3.80 (s, 3H), 4.16 (dd, 1H, J = 8.2 Hz, 6.4 Hz), 6.70 (S, 1H), 6.78 (s, 1H), 7.06-7.18 (m, 4H) |
| 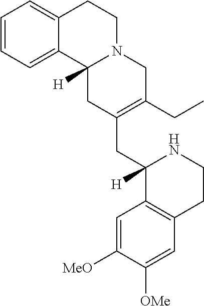 [SMILES: CCC1=C(C[C@@H]2NCCc3cc(OC)c(OC)cc32)C[C@@H]2c3ccccc3CCN2C1] | UCL-ONY-002 | (R,S) | 64.8% [M + H]⁺ 419.4 | ¹H NMR (MeOD, 270 MHz) δ ppm 1.05 (t, 3H, J = 7.6 Hz), 2.05-2.40 (m, 3H), 2.44-2.88 (m, 7H), 2.92-3.19 (m, 4H), 3.20-3.29 (m, 1H), 3.32-3.39 (m, 1H), 3.49 (dd, 1H, J = 10.9 Hz, 4.3 Hz), 3.79 (s, 3H), 3.80 (s, 3H), 4.16 (dd, 1H, J = 8.2 Hz, 6.4 Hz), 6.70 (s, 1H), 6.78 (s, 1H), 7.06-7.18 (m, 4H) |
| 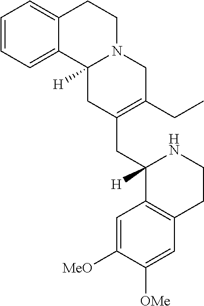 [SMILES: CCC1=C(C[C@@H]2NCCc3cc(OC)c(OC)cc32)C[C@H]2c3ccccc3CCN2C1] | UCL-ONY-003 | (S,S)/(S,R) mixture 1:0.2 | 98.0%* [M + H]⁺ 419.4 *combined purity of diastereomers | ¹H NMR (MeOD, 270 MHz) δ ppm 1.00 (t, 3H, J = 7.6 Hz), 1.96-2.31 (m, 3H), 2.46-2.98 (m, 8H), 3.02-3.25 (m, 4H), 3.35 (bd, 1H, J = 15.8 Hz), 3.44-3.50 (m, 0.15Heq), 3.50-3.59 (m, 0.85Heq), 3.79 (s, 3H), 3.81 (s, 3H), 4.04-4.14 (m, 1H), 6.68 (s, 1H), 6.78 (s, 1H), 7.05-7.22 (m, 3H), 7.23-7.31 (m, 1H) |
| 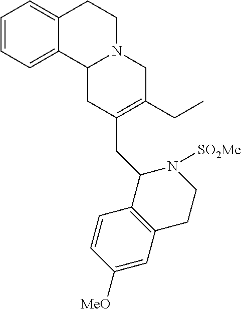 [SMILES: CCC1=C(CC2c3ccc(OC)cc3CCN2S(C)(=O)=O)CC2c3ccccc3CCN2C1] | UCL-ONY-004 | Racemic mixture | 72.1% [M + H]⁺ 467.5 | ¹H NMR (MeOD, 270 MHz) δ ppm 0.94 (t, 1.5Heq, J = 7.6 Hz), 1.02 (t, 1.5Heq J = 7.6 Hz), 1.16-1.30 (m, 1H), 1.35-1.88 (m, 4H), 1.90-2.35 (m, 4H), 2.37-2.61 (m, 3H), 2.80 (2x s, 3Heq), 2.83-2.95 (m, 2H), 3.43 (dd, 0.5Heq, J = 10.8 Hz, 2.7 Hz), 3.53 (dd, 0.5Heq, J = 10.8 Hz, 2.7 Hz), 3.65-3.73 (m, 1H), 3.77 (s, 3H), 3.75-3.87 (m, 1H), 4.88-4.98 (m, 1H), 6.61-6.66 (m, 1H), 6.72-6.78 (m, 1H), 6.97-7.25 (m, 5H) |

### Example 9—HIF Inhibition Using Analogues of Compound I (Batch 2)

Referring to FIG. 11, there is shown the compound represented by formula I (i.e. “HIF-Inhib1”), and three enantiomers (S, R-“UCL-ONY-001”; R, S-“UCL-ONY-002”; and S, S-“UCL-ONY-003”), and a racemic analogue (“UCL-ONY-004”). These compounds were then tested for their HIF activity using the luciferase assay, and the data are shown in FIG. 12.

FIG. 12A is a graph showing the percentage inhibition of luciferase activity in U2OS-HRE cells treated with the compounds shown in FIG. 11. The compounds were dosed at 1 μM and incubated in 1% 02 for 16 hours. FIG. 12B shows Western blot analysis of U2OS-HRE cells treated with compounds as in FIG. 1C to show inhibitory effects on HIF-1α, phosphorylated and total eIF2α protein levels. Tubulin was used as a loading control.

As can be seen, the S, R enantiomer (“UCL-ONY-001”) exhibits similar inhibitory activity as HIF-Inhib1 in the U2OS_HRE luciferase cells, while UCL-ONY-002, 003 and 004 are inactive. The S, R enantiomer is: (S)-2-(((R)-6,7-dimethoxy-1,2,3,4-tetrahydroisoquinolin-1-yl)methyl)-3-ethyl-1,6,7,11b-tetrahydro-4H-pyrido[2,1-a]isoquinoline. Therefore, the S, R enantiomer (“UCL-ONY-001”) is believed to be responsible for the activity. This is further confirmed by the mechanism of action analysis shown in FIG. 12B, where the inventors have found that the S,R enantiomer (“UCL-ONY-001”) has similar inhibitory activity to HIF-Inhib1 in blocking HIF1α protein induction and eIF-2α phosphorylation.

1. A method of treating, preventing or ameliorating a disease characterised by abnormal levels of hypoxia-inducible factor (HIF) activity, the method comprising administering, to a subject in need of such treatment, a therapeutically effective amount of a compound of formula (I):— 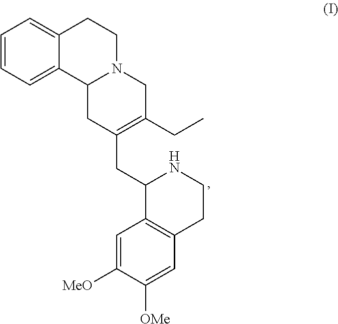 [SMILES: CCC1=C(CC2NCCc3cc(OC)c(OC)cc32)CC2c3ccccc3CCN2C1]
or a functional analogue, or derivative, or pharmaceutically acceptable salt or solvate thereof.

2. The method according to claim 1, wherein the compound (I) is the S, R enantiomer.

3. The method according to claim 1, wherein the compound, or a functional analogue, or derivative, or pharmaceutically acceptable salt or solvate thereof, inhibits the hypoxia-inducible factor (HIF) transcriptional complex.

4. The method according to claim 1, wherein the compound, or a functional analogue, or derivative, or pharmaceutically acceptable salt or solvate thereof, reduces or blocks expression of hypoxia-inducible factor-1 alpha (HIF-1α).

5. The method according to claim 1, wherein the compound, or a functional analogue, or derivative, or pharmaceutically acceptable salt or solvate thereof, reduces or blocks expression of vascular endothelial growth factor (VEGF).

6. The method according to claim 1, wherein the compound, or a functional analogue, or derivative, or pharmaceutically acceptable salt or solvate thereof, reduces or blocks eIF-2α phosphorylation.

7-12. (canceled)

13. A pharmaceutical composition comprising a compound of formula (I):— 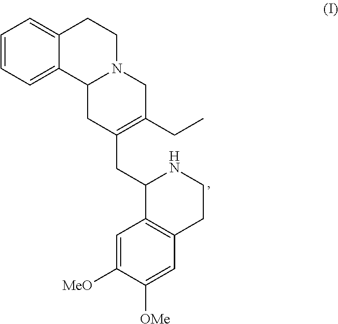 [SMILES: CCC1=C(CC2NCCc3cc(OC)c(OC)cc32)CC2c3ccccc3CCN2C1]
or a functional analogue, or derivative, or pharmaceutically acceptable salt or solvate thereof, and a pharmaceutically acceptable vehicle.

14. A composition according to claim 13, wherein the composition is an anti-cancer pharmaceutical composition.

15. A process for making the composition according to claim 13, the process comprising contacting a therapeutically effective amount of a compound of formula (I), or a functional analogue, or derivative, or pharmaceutically acceptable salt or solvate thereof, and a pharmaceutically acceptable vehicle.

16. A composition according to claim 13, wherein the composition is for treating prostate cancer.

17. A hypoxia-inducible factor (HIF) pathway inhibitor comprising a compound of formula (I):— 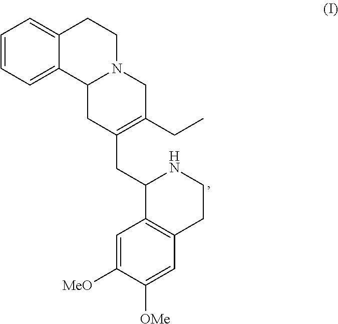 [SMILES: CCC1=C(CC2NCCc3cc(OC)c(OC)cc32)CC2c3ccccc3CCN2C1]
or a functional analogue, or derivative, or pharmaceutically acceptable salt or solvate thereof.

18. The method according to claim 1, wherein the disease is cancer.

19. The method according to claim 18, wherein the cancer is prostate cancer.

20. The method according to claim 1, wherein the compound, or a functional analogue, or derivative, or pharmaceutically acceptable salt or solvate thereof, is used to treat hepatitis C or hepatoma cell migration.

21. The method according to claim 1, wherein the compound, or a functional analogue, or derivative, or pharmaceutically acceptable salt or solvate thereof, is used to treat a tumour or cancer-based disease where HIF is constitutively upregulated and HIF-α protein is overexpressed.
